(19) United States
(12) Patent Application Publication
(10) Pub. No.: US 2018/0193365 A1
(43) Pub. Date: Jul. 12, 2018

(54) USE OF HYDROGEN SULFIDE TO PREVENT LENS OPACIFICATION

(71) Applicant: Creighton University, Omaha, NE (US)

(72) Inventor: Catherine Opere, Omaha, NE (US)

(21) Appl. No.: 15/866,083

(22) Filed: Jan. 9, 2018

Related U.S. Application Data
(60) Provisional application No. 62/443,872, filed on Jan. 9, 2017.

(51) Int. Cl. A61K 31/675; A61P 27/12; A61K 31/10; A61K 9/00

(52) U.S. Cl. CPC A61K 31/675; A61K 9/0048; A61K 31/10; A61P 27/12

(57) ABSTRACT

Provided herein are hydrogen sulfide-releasing compounds useful for reducing and/or preventing the formation of cataracts in a subjects.

### CROSS-REFERENCE TO RELATED APPLICATIONS

This application claims the benefit of U.S. Provisional Application Ser. No. 62/443,872, filed Jan. 9, 2017, the disclosure of which is incorporated herein by reference in its entirety.

### TECHNICAL FIELD

The present disclosure relates to the use of hydrogen sulfide-releasing compounds for reducing and/or preventing the formation of cataracts in a subject.

### BACKGROUND

The term “cataract” refers to gradual clouding of the lens, leading to impairment of vision and blindness in a subject, if untreated. Cataracts are one of the leading causes of vision impairment worldwide, particularly among the geriatric population. In the United States, the number of people suffering from cataracts is estimated to double from 24.4 million in 2010 to approximately 50 million by the year 2050 (see e.g., Cataracts. The National Institutes of Health (NIH), National Eye Institute (NEI), https://nei.nih.gov/eyedata/cataract#5). Currently, there is no pharmacological intervention for treatment of cataracts and surgery is the mainstay of therapy. Thus, pharmacological approaches to treatment of cataracts may play a particular role in regions of the world surgical options are limited.

### SUMMARY

The present application provides, inter alia, a method of preventing a cataract in a subject in need thereof, comprising administering to the subject a therapeutically effective amount of a hydrogen sulfide-releasing compound provided herein, or a pharmaceutically acceptable salt thereof.

The present application further provides a method of delaying the formation of a cataract in a subject in need thereof, comprising administering to the subject a therapeutically effective amount of a hydrogen sulfide-releasing compound provided herein, or a pharmaceutically acceptable salt thereof.

In some embodiments, the methods provided herein comprise delaying opacification of the eye or reducing the rate of opacification of the eyes. In some embodiments, the opacification comprises H₂O₂-induced opacification.

The present application further provides a method of reducing oxidative stress in the eye of a subject in need thereof, comprising administering to the subject a therapeutically effective amount of a hydrogen sulfide-releasing compound provided herein, or a pharmaceutically acceptable salt thereof.

The present application further provides a method of monitoring the formation of a cataract in a subject in need thereof, comprising:

i) administering to the subject a therapeutically effective amount of a hydrogen sulfide-releasing compound provided herein, or a pharmaceutically acceptable salt thereof; and

ii) imaging an eye of the subject with a suitable imaging technique.

In some embodiments, the methods provided herein further comprise imaging an eye of the subject with a suitable imaging technique prior to step i). In some embodiments, the imaging is performed using a photographic imaging technique, a digital imaging technique, or any combination thereof.

In some embodiments, the methods provided herein comprise locally administering the hydrogen sulfide-releasing compound, or pharmaceutically acceptable salt thereof, to an eye of the subject. In some embodiments, the hydrogen sulfide-releasing compound provided herein, or pharmaceutically acceptable salt thereof, is administered via topical administration.

In some embodiments, the hydrogen sulfide-releasing compound provided herein, or pharmaceutically acceptable salt, is administered in the form of a pharmaceutical composition comprising the compound, or pharmaceutically acceptable salt thereof, and at least one additional pharmaceutically acceptable carrier.

In some embodiments, the subject has been identified as exhibiting one or more symptoms associated with a cataract. In some embodiments, the subject has been identified as exhibiting one or more risk factors associated with a cataract selected from the group consisting of diabetes, obesity, hypertension, ocular trauma, myopia, ocular inflammatory disease, and chronic use of corticosteroids.

In some embodiments, the hydrogen sulfide-releasing compound is selected from the group consisting of morpholin-4-ium (4-methoxyphenyl)(morpholino)phosphinodithioate, diallyl trisulfide, M₂S, and MHS, wherein M is an alkali metal. In some embodiments, the hydrogen sulfide-releasing compound is selected from the group consisting of morpholin-4-ium (4-methoxyphenyl)(morpholino)phosphinodithioate and diallyl trisulfide. In some embodiments, M is sodium or potassium.

In some embodiments, the methods provided herein are performed in combination with a surgical procedure for treating a cataract. In some embodiments, the methods provided herein further comprise administering one or more additional therapeutic agents useful for the treatment of a cataract to the subject.

In some embodiments, the methods provided herein comprise reducing and/or reversing the degradation of glutathione (GSH) In some embodiments, the methods provided herein comprise reducing and/or reversing the degradation of superoxide dismutase (SOD) enzyme in the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIGS. 1A-1B show the effect of a control solution (DMEM buffer), morpholin-4-ium (4-methoxyphenyl)(morpholino)phosphinodithioate (GYY 4137; 10⁻⁴ M), H₂O₂ (50 mM), and a mixture of GYY 4137/H₂O₂ on bovine lens opacification (t=0). FIG. 1A shows % transmittance and FIG. 1B shows a representative photograph of the treated bovine lenses.

FIGS. 2A-2B show the effect of a control solution (DMEM buffer), GYY 4137 (10⁻⁴ M), H₂O₂ (50 mM), and a mixture of GYY 4137/H₂O₂ on bovine lens opacification (t=12 h). FIG. 2A shows % transmittance and FIG. 2B shows a representative photograph of the treated bovine lenses.

FIGS. 3A-3B show the effect of a control solution (DMEM buffer), GYY 4137 (10⁻⁴ M), H₂O₂ (50 mM), and a mixture of GYY 4137/H₂O₂ on bovine lens opacification (t=36 h). FIG. 3A shows % transmittance and FIG. 3B shows a representative photograph of the treated bovine lenses.

FIGS. 4A-4B show the effect of a control solution (DMEM buffer), GYY 4137 (10⁻⁴ M), H₂O₂ (50 mM), and a mixture of GYY 4137/H₂O₂ on bovine lens opacification (t=48 h). FIG. 4A shows % transmittance and FIG. 4B shows a representative photograph of the treated bovine lenses.

FIGS. 5A-5B show the effect of a control solution (DMEM buffer), GYY 4137 (10⁻⁴ M), H₂O₂ (50 mM), and a mixture of GYY 4137/H₂O₂ on bovine lens opacification (t=96 h). FIG. 5A shows % transmittance and FIG. 5B shows a representative photograph of the treated bovine lenses.

FIGS. 6A-6B show the effect of a control solution (DMEM buffer), GYY 4137 (10⁻⁴M), H₂O₂ (50 mM), and a mixture of GYY 4137/H₂O₂ on bovine lens opacification (t=108 h). FIG. 6A shows % transmittance and FIG. 6B shows a representative photograph of the treated bovine lenses.

FIGS. 7A-7B show the effect of a control solution (DMEM buffer), GYY 4137 (10⁻⁴M), H₂O₂ (50 mM), and a mixture of GYY 4137/H₂O₂ on bovine lens opacification (t=120 h). FIG. 7A shows % transmittance and FIG. 7B shows a representative photograph of the treated bovine lenses.

FIG. 8 shows the percent transmittance at 350 nm of untreated bovine lenses and bovine lenses treated with GYY 4137 (10⁻⁴M), H₂O₂ (50 mM), and a mixture of GYY 4137/H₂O₂.

FIG. 9 shows the percent transmittance at 500 nm of untreated bovine lenses and bovine lenses treated with GYY 4137 (10⁻⁴M), H₂O₂ (50 mM), and a mixture of GYY 4137/H₂O₂.

FIG. 10 shows degradation of DMEM-cultured bovine lenses after 120 hours. Light transmittance (at 420 nm) elicited by DMEM (0 hour), DMEM (120 hours) or ascorbic acid (AA; 3 mM and 10 mM). Vertical bars represent means±SEM. Number of observations is in parenthesis. ++++p<0.0001 significantly different from untreated lenses (DMEM; 0 hours), significantly different from untreated lenses (DMEM; 120 hours).

FIG. 11 shows the effect of diallyl trisulfide (DATS) on cultured bovine lens degradation for 120 hours. #### P<0.0001, significantly different from untreated lenses (DMEM; 120 hours); 6-12 number of observations per concentration.

FIG. 12 shows the effect of DATS on cultured bovine lens degradation after 120 hours. Light transmittance (at 420 nm) elicited by DMEM (120 hours), ascorbic acid (AA; 10 mM) or DATS (10⁻⁷M to 10⁻⁴M). Vertical bars represent means±SEM. Number of observations is in parenthesis. ̂̂̂<0.0001, significantly different from untreated lenses (DMEM; 120 hours).

FIG. 13 shows a qualitative assessment of the effect of DATS on lens opacity after 120 hours. Photographic depiction of the effect of DATS on cultured bovine lenses, in vitro is shown. Two representative lenses provided for each treatment. Top Panel, DMEM (0 hours), DMEM (120 hours) and ascorbic acid (10 mM; 120 hours); Bottom panel, DATS (10⁻⁷M-10⁻⁴M) following 120 hours of treatment.

FIG. 14 shows the effect of GYY 4137 on cultured bovine lens degradation for 120 hours. #### P<0.0001, significantly different from untreated lenses (DMEM; 120 hours); 6-12 number of observations per concentration.

FIG. 15 shows the effect of GYY 4137 on cultured bovine lens degradation after 120 hours. Light transmittance (at 420 nm) elicited by DMEM (120 hours), ascorbic acid (AA; 10 nmM) or GYY 4137 (10⁻⁷M-10⁻⁵M). Vertical bars represent means±SEM. Number of observations is in parenthesis. #### P<0.0001, significantly different from untreated lenses (DMEM; 120 hours).

FIG. 16 shows a qualitative assessment of the effect of GYY 4137 on lens opacity after 120 hours. Photographical depiction of the effect of GYY 4137 on cultured bovine lenses, in vitro. Two representative lenses provided for each treatment. Top Panel, DMEM (0 hours), DMEM (120 hours) and ascorbic acid (10 mM; 120 hours); Bottom panel, GYY 4137 (10⁻⁷M-10⁻⁴M) following 120 hours of treatment.

FIG. 17 shows hydrogen peroxide (H₂O²)-induced degradation of DMEM-cultured bovine lenses after 120 hours. Light transmittance (at 420 nm) elicited by control (DMEM, 0-hour), H₂O₂ (50 mM) or ascorbic acid (AA; 3 mM and 10 mM) in presence of H₂O₂. Vertical bars represent means±SEM. Number of observations is in parenthesis. ###p<0.0001 significantly different from untreated lenses (DMEM; 0 hours).

FIG. 18 shows the effect of DATS on hydrogen peroxide (H₂O₂)-induced lens degradation in cultured bovine lenses over 120 hours period. ++++P<0.0001, significantly different from untreated lenses H₂O₂ (50 mM) 120 hours. 6-12 number of observations per concentration.

FIG. 19 shows the effect of DATS on hydrogen peroxide (H₂O₂)-induced lens degradation after 120 hours. Light transmittance (at 420 nm) elicited by H₂O₂ (50 mM); DATS (10⁻⁷M to 10⁻⁴M) in the presence of H₂O₂. Vertical bars represent means±SEM. Number of observations is in parenthesis. ++++P<0.0001, significantly different from untreated lenses (H₂O₂).

FIG. 20 shows a qualitative assessment of the effect of DATS on hydrogen peroxide (H₂O₂; 50 mM)-induced lens opacity after 120 hours. Photographical depiction of the effect of DATS on cultured bovine lenses, in vitro. Two representative lenses provided for each treatment. Top Panel, DMEM (0 hours), H₂O₂ (50 mM; 120 hours) and ascorbic acid (AA; 3 mM; 120 hours); Bottom panel, DATS (10⁻⁷M-10⁻⁴M) in presence of H₂O₂ after 120 hours.

FIG. 21 shows the effect of GYY 4137 on hydrogen peroxide (H₂O₂)-induced lens degradation in cultured bovine lenses over 120 hours period. ++++P<0.0001 significantly different from lenses cultured in H₂O₂ (50 mM). E*: transmittance was lower than H₂O₂ treated lenses (at 6 hours 10⁻⁶M-p>0.5).

FIG. 22 shows the effect of GYY 4137 on hydrogen peroxide (H₂O₂)-induced lens degradation after 120 hours. Light transmittance (at 420 nm) elicited by H₂O₂ (50 mM); GYY 4137 (10⁻⁷M to 10⁻⁴M) in the presence of H₂O₂. Vertical bars represent means±SEM. Number of observations is in parenthesis. ***P<0.0001, significantly different from H₂O₂-treated lenses.

FIG. 23 shows a qualitative assessment of the effect of GYY 4137 on hydrogen peroxide (H₂O₂; 50 mM)-induced lens opacity after 120 hours. Photographical depiction of the effect of GYY 4137 on cultured bovine lenses, in vitro. Two representative lenses provided for each treatment. Top Panel, DMEM (0 hours), H₂O₂ (50 mM; 120 hours) and ascorbic acid (AA; 3 mM; 120 hours); Bottom panel, GYY 4137 (10⁻⁷M-10⁻⁴M) in presence of H₂O₂ after 120 hours.

FIG. 24 shows the effect of hydrogen sulfide (H₂S) donors, DATS (10⁻⁶M) and GYY 4137 (10⁻⁷M) on glutathione (GSH) content in H₂O₂-treated cultured bovine lenses after a duration of 120 hours. Control (0 hours); control (120 hours); H₂S donors in absence and presence of H₂O₂ (50 mM). Vertical bars represent means±SEM (n=5). ****P<0.0001 compared to untreated lenses at t=0 h, #### P<0.0001 compared to untreated lenses at t=120 hours, ++++P<0.0001 compared to H₂O₂ treated lenses t=120 hours.

FIG. 25 shows the effect of hydrogen sulfide (H₂S) donors, DATS (10⁻⁶M) and GYY 4137 (10⁻⁷M) on superoxide dismutase (SOD) enzyme in H₂O₂-treated cultured bovine lenses after a duration of 120 hours. Control (0 hours); control (120 hours); H₂S donors in absence and presence of H₂O₂ (50 mM). Vertical bars represent means±SEM (n=5). ****P<0.0001 compared to untreated lenses at t=0 h, #### P<0.0001 compared to untreated lenses at t=120 hours, ++++P<0.0001 compared to H₂O₂ treated lenses t=120 hours.

### DETAILED DESCRIPTION

Hydrogen sulfide (H₂S) is an odiferous water-soluble gas that has been known as an industrial and environmental pollutant (see e.g., Wagner et al, Crit. Care, 2009, 13:213). Recently, H₂S has emerged as the third signaling molecule and gasotransmitter in mammalian tissue (after nitric oxide and carbon monoxide) (see e.g., Wagner et al, Crit. Care, 2009, 13:213). H₂S is endogenously synthesized from the sulfur containing amino acid, L-cysteine by the activity of either of two pyridoxal 5′-phosphate (vitamin B6)-dependent enzymes, cystathionine-β-synthase (CBS) or cystathionine-γ-lyase (CSE) enzymes, and 3-mercaptopyruvate sulfurtransferase (3MST) along with cysteine aminotransferase (see e.g., Cooper et al, Ann. Rev. Biochem. 1983, 52:187-222; Shibuya et al, J. Biol. Chem. 2009, 146, 623-626; and Shibuya et al, Antioxidants & Redox Signaling, 2009, 11:703-714). In ocular tissues, H₂S has been shown to reduce intraocular pressure in the anterior uvea in normotensive and glaucomatous animals (see e.g., U.S. Pat. No. 8,092,838; Salvi et al, J. Ocul. Pharmacol. Ther. 2016, 32:371-375; and Perrino et al, Bioorganic Med. Chem. Lett. 2009, 19:1639-1642) and attenuate retinal degeneration in vitro and in vivo (see e.g., Sakamoto et al, Exp. Eye Res. 2014, 120:90-96; and Osborne et al, Invest. Ophthalmol. & Vis. Sci. 2012, 51:284-294).

Oxidative stress has been implicated in the pathogenesis of cataract development in the mammalian eye (see e.g., Spector A. FASEB J. 1995, 9:1173-1182; Lou et al, Prog. Retin. Eye Res. 2003, 22:657-682; and Wu et al, J. Biol. Chem. 2014, 289(52):36125-36139). Compared to normal subjects, hydrogen peroxide (H₂O₂) content was found to be significantly elevated in aqueous humor and lenses of cataract patients (see e.g., Spector A. FASEB J. 1995, 9:1173-1182). Moreover, the peroxide insult mimicked the pattern of damage associated with some forms of human cataract (see e.g., Spector A. FASEB J. 1995, 9:1173-1182).

### Methods of Use

The present application provides method of preventing a cataract in a subject in need thereof, comprising administering to the subject a hydrogen sulfide-releasing compound provided herein, or a pharmaceutically acceptable salt thereof.

The present application further provides a method of delaying the formation of a cataract in a subject in need thereof, comprising administering to the subject a hydrogen sulfide-releasing compound provided herein, or a pharmaceutically acceptable salt thereof.

The present application further provides a method of reducing oxidative stress in the eye of a subject in need thereof, comprising administering to the subject a hydrogen sulfide-releasing compound provided herein, or a pharmaceutically acceptable salt thereof.

The present application further provides a method of reducing the rate of formation of a cataract in a subject in need thereof, comprising administering to the subject a hydrogen sulfide-releasing compound provided herein, or a pharmaceutically acceptable salt thereof.

The present application further provides a method of treating a cataract in a subject in need thereof, comprising administering to the subject a hydrogen sulfide-releasing compound provided herein, or a pharmaceutically acceptable salt thereof.

In some embodiments, the methods provided herein comprise delaying and/or reducing the progression (e.g., growth and/or expansion) of a cataract in the subject. In some embodiments, the methods provided herein comprise delaying and/or reducing the rate of opacification of the eye and/or reducing the rate of opacification of the eyes. In some embodiments, the methods provided herein comprise delaying opacification of the eye. In some embodiments, the methods provided herein comprise reducing the rate of opacification of the eye. In some embodiments, the opacification comprises H₂O₂-induced opacification.

The present application further provides a method of monitoring the formation of a cataract in a subject in need thereof, comprising:

i) administering to the subject a hydrogen sulfide-releasing compound provided herein, or a pharmaceutically acceptable salt thereof; and

ii) imaging an eye of the subject with a suitable imaging technique.

The present application further provides a method of monitoring opacification in a subject in need thereof, comprising:

i) administering to the subject a hydrogen sulfide-releasing compound provided herein, or a pharmaceutically acceptable salt thereof; and

ii) imaging an eye of the subject with a suitable imaging technique.

In some embodiments, the methods provided herein further comprise imaging an eye of the subject with a suitable imaging technique prior to step i). In some embodiments, the imaging is performed using a photographic imaging technique, a digital imaging technique, slit-lamp biomicroscopy, or any combination thereof.

In some embodiments, the methods provided herein further comprise:

iii) administering the hydrogen sulfide-releasing compound one or more additional times after the imaging of step ii).

In some embodiments, the methods provided herein further comprise:

iii) administering the hydrogen sulfide-releasing compound one or more additional times after the imaging of step ii); and

iv) imaging an eye of the subject with a suitable imaging technique.

In some embodiments, the methods provided herein further comprise comparing the image of step iv) and the image of step ii) to qualitatively and/or quantitatively determine the differences between the images.

In some embodiments, the methods provided herein further comprise waiting a time sufficient to allow the hydrogen sulfide-releasing compound, or a pharmaceutically acceptable salt thereof, to accumulate at a cell or tissue site (e.g., a cell or tissue site in a subject) associated with the cataract and/or the site of opacification, prior to the imaging of step ii) and/or step iv).

In some embodiments, the time sufficient to allow the hydrogen sulfide-releasing compound to accumulate at a cell or tissue site is from about 30 seconds to about 7 days, for example, about 30 seconds to about 5 days, about 30 seconds to about 3 days, about 30 seconds to about 24 hours, about 30 seconds to about 12 hours, about 30 seconds to about 6 hours, about 30 seconds to about 2 hours, about 30 seconds to about 1 hour, about 1 hour to about 5 days, about 1 hour to about 5 days, about 1 hour to about 3 days, about 1 hour to about 24 hours, about 1 hour to about 12 hours, about 1 hour to about 6 hours, about 1 hour to about 2 hours, about 2 hours to about 5 days, about 2 hours to about 5 days, about 2 hours to about 3 days, about 2 hours to about 24 hours, about 2 hours to about 12 hours, about 2 hours to about 6 hours, about 6 hours to about 5 days, about 6 hours to about 5 days, about 6 hours to about 3 days, about 6 hours to about 24 hours, about 6 hours to about 12 hours, about 12 hours to about 5 days, about 12 hours to about 5 days, about 12 hours to about 3 days, about 12 hours to about 24 hours, about 24 hours to about 5 days, about 24 hours to about 5 days, about 24 hours to about 3 days, or about 3 days to about 5 days.

The present application further provides a method of increasing the amount of glutathione (GSH) and/or superoxide dismutase (SOD) enzyme in a cell or tissue, comprising contacting the cell or tissue with a hydrogen sulfide-releasing compound provided herein, or a pharmaceutically acceptable salt thereof. In some embodiments, the method is a method of increasing the amount of glutathione in the cell or tissue. In some embodiments the method is a method of increasing the amount of superoxide dismutase in the cell or tissue. In some embodiments, the method is a method of increasing the amount of glutathione and superoxide dismutase in the cell or tissue.

The present application further provides a method of increasing the rate of formation of glutathione (GSH) and/or superoxide dismutase (SOD) enzyme in a cell or tissue, comprising contacting the cell or tissue with a hydrogen sulfide-releasing compound provided herein, or a pharmaceutically acceptable salt thereof. In some embodiments, the method is a method of increasing rate of formation of glutathione in the cell or tissue. In some embodiments the method is a method of increasing the rate of formation of superoxide dismutase in the cell or tissue. In some embodiments, the method is a method of increasing rate of formation of glutathione and superoxide dismutase in the cell or tissue. In some embodiments, the method is an in vitro method. In some embodiments, the method is an in vivo method.

The present application further provides a method of increasing the amount of glutathione (GSH) and/or superoxide dismutase (SOD) enzyme in a subject, comprising administering to the subject a hydrogen sulfide-releasing compound provided herein, or a pharmaceutically acceptable salt thereof. In some embodiments, the method is a method of increasing the amount of glutathione in the eye of a subject. In some embodiments the method is a method of increasing the amount of superoxide dismutase in eye of a subject. In some embodiments, the method is a method of increasing the amount of glutathione and superoxide dismutase in the eye of a subject.

The present application further provides a method of increasing the rate of formation of glutathione (GSH) and/or superoxide dismutase (SOD) enzyme in a subject, comprising contacting the cell or tissue with a hydrogen sulfide-releasing compound provided herein, or a pharmaceutically acceptable salt thereof. In some embodiments, the method is a method of increasing rate of formation of glutathione in the eye of the subject. In some embodiments the method is a method of increasing the rate of formation of superoxide dismutase in the eye of the subject. In some embodiments, the method is a method of increasing rate of formation of glutathione and superoxide dismutase in the eye of the subject.

The present application further provides a method of reducing and/or reversing the degradation of glutathione (GSH) and/or superoxide dismutase (SOD) enzyme in a cell or tissue, comprising contacting the cell or tissue with a hydrogen sulfide-releasing compound provided herein, or a pharmaceutically acceptable salt thereof. In some embodiments, the method is a method of reducing and/or reversing the degradation of glutathione in the cell or tissue. In some embodiments the method is a method of reducing and/or reversing the degradation of superoxide dismutase in the cell or tissue. In some embodiments, the method is a method of reducing and/or reversing the degradation of glutathione and superoxide dismutase in the cell or tissue.

The present application further provides a method of reducing and/or reversing the degradation of glutathione (GSH) and/or superoxide dismutase (SOD) enzyme in a subject, comprising administering to the subject a hydrogen sulfide-releasing compound provided herein, or a pharmaceutically acceptable salt thereof. In some embodiments, the method is a method of reducing and/or reversing the degradation of glutathione in the eye of the subject. In some embodiments the method is a method of reducing and/or reversing the degradation of superoxide dismutase in the eye of the subject. In some embodiments, the method is a method of reducing and/or reversing the degradation of glutathione and superoxide dismutase in the eye of the subject.

In some embodiments, the methods provided herein comprise locally administering the hydrogen sulfide-releasing compound, or pharmaceutically acceptable salt thereof, to an eye of the subject. In some embodiments, the administration is topical administration (e.g. ophthalmic administration, administration to mucous membranes, and the like), oral, or parenteral administration (e.g., intravenous, intraarterial, subcutaneous, intraperitoneal, intramuscular or injection or infusion, intracranial, intrathecal, intraventricular administration, and the like). In some embodiments, administration is topical administration. In some embodiments, administration is ophthalmic administration. In some embodiments, administration is parenteral administration. In some embodiments, administration is oral administration.

As used herein, the term “subject,” refers to any animal, including mammals. Example subjects include, but are not limited to, mice, rats, rabbits, dogs, cats, swine, cattle, sheep, horses, primates, and humans. In some embodiments, the subject is a human. In some embodiments, the method comprises administering to the subject a therapeutically effective amount of a hydrogen sulfide-releasing compound provided herein, or a pharmaceutically acceptable salt thereof.

In some embodiments, the subject has been identified as exhibiting one or more symptoms associated with a cataract. Exemplary symptoms include, but are not limited to, ocular lens clouding, hazy vision, blurred vision, glaring, observing a halo near bright light, monocular diplopia (i.e., multiple images of same object), loss of lens transparency, lens discoloration (e.g. yellow and/or brown discoloration due to precipitation of lens proteins).

In some embodiments, the subject has been identified as having one or more symptoms associated with a cataract using a technique selected from the group consisting of a visual acuity test, a glare test, a pupillary function assessment, an ocular alignment assessment, and a motility assessment, or any combination thereof.

In some embodiments, the subject has been identified as exhibiting one or more risk factors associated with a cataract. For example, the American Association of Ophthalmology (AOA) has identified several risk factors for cataracts, including diabetes, obesity, hypertension, ocular trauma, myopia, ocular inflammatory disease, and chronic use of corticosteroids (see e.g., Olson et al, Ophthalmology, 2017, 124(2):P1-P119). In some embodiments, the subject has been identified as exhibiting one or more risk factors associated with a cataract selected from the group consisting of diabetes, obesity, hypertension, ocular trauma, myopia, ocular inflammatory disease, and chronic use of corticosteroids.

Use of glucocorticoids particularly renders patients susceptible to cataractogenesis. Clinically, glucocorticoids have wide therapeutic application, ranging from treatment of cancer, immunological conditions, and metabolic disorders. However, chronic use of glucocorticoids is strongly associated with development of ocular toxicities, including cataracts (see e.g., Abadia et al, Drugs Aging, 2016, 33:639; and Bandello et al, Dev. Ophthalmol. Basil, Karger, 2017, 60:78-90). Thus, use of H₂S releasing compounds in populations that require glucocorticoid therapy could be useful for improving patient outcomes.

In some embodiments, the subject is about 12 months of age or younger. In some embodiments, the subject is from about 12 months to about 100 years of age, for example, about 12 months to about 70 years, about 12 months to about 50 years, about 12 months to about 30 years, about 12 months to about 18 years, about 18 years to about 100 years, about 18 years to about 70 years, about 18 years to about 50 years, about 18 years to about 30 years, about 30 years to about 100 years, about 30 years to about 70 years, about 30 years to about 50 years, about 50 years to about 100 years, about 50 years to about 70 years, or about 70 years to about 100 years of age. In some embodiments, the subject is about 40 years of age or older, about 50 years of age or older, about 60 years of age or older, about 70 years of age or older, about 80 years of age or older, or about 90 years of age or older.

The prevalence of cataracts increases with aging, rendering the geriatric population highly susceptible to this ocular condition (see e.g., Olson et al, Ophthalmology, 2017, 124(2):P1-P119). In some embodiments, the subject is from about 40 to about 100 years of age, for example, about 40 to about 90, about 40 to about 80, about 40 to about 70, about 40 to about 60, about 40 to about 50, about 50 to about 90, about 50 to about 80, about 50 to about 70, about 50 to about 60, about 60 to about 90, about 60 to about 80, about 60 to about 70, about 70 to about 90, about 70 to about 80, or about 80 to about 90 years of age.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

In some embodiments, the hydrogen sulfide-releasing compound is selected form the group of compounds provided in Table 1.

TABLE 1

| H₂S Releasing | |
|---|---|
| Compound or Class | |
| of Compounds | Representative Chemical Structure |
| Hydrogen sulfide | H₂S |
| (gas) | |
| Sulfide salts | NaHS, Na₂S, CaS, MoS₄²⁻ salts (e.g., H₈N₂MoS₄) |
| Natural sulfide releasing compounds | 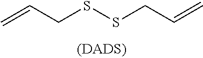 |
| | 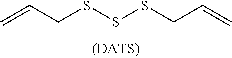 |
| | 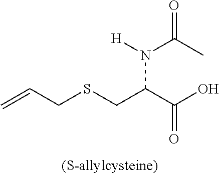 |
| SG-1002 | 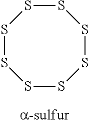 |
| Lawesson's Reagent | 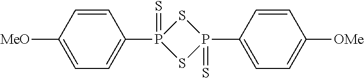 [SMILES: COc1ccc(P2(=S)SP(=S)(c3ccc(OC)cc3)S2)cc1] |
| GYY4137 | 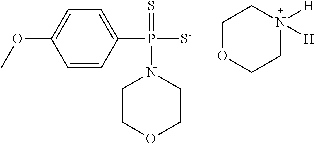 |
| Thioamide | 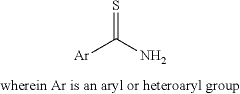 |
| Isothiocyanate | 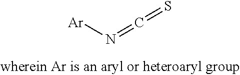 |
| 1,2-Dithiole-3- thiones (DTTs) | 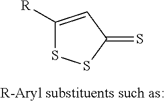 |
| | 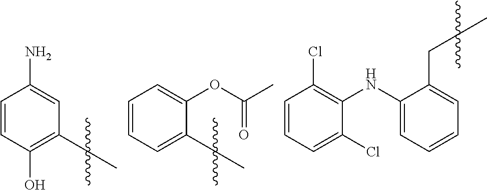 |
| Tertiary perthiol-based H₂S releasing compounds | 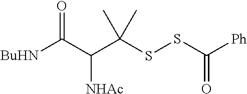 |
| Perthiol-based H₂S releasing compounds | 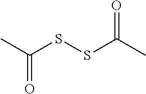 |
| Ketoprofenate caged H₂S releasing compounds | 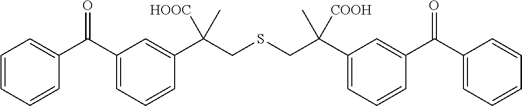 [SMILES: CC(CSCC(C)(C(=O)O)c1cccc(C(=O)c2ccccc2)c1)(C(=O)O)c1cccc(C(=O)c2ccccc2)c1] |
| Xanthone caged H₂S releasing compounds | 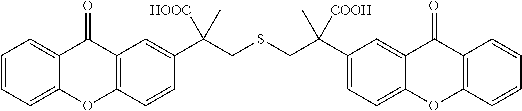 [SMILES: CC(CSCC(C)(C(=O)O)c1ccc2oc3ccccc3c(=O)c2c1)(C(=O)O)c1ccc2oc3ccccc3c(=O)c2c1] |
| Carbonyl sulfide based releasing compounds (e.g., benzyl thiocarbamates) | 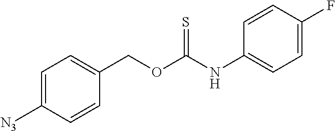 [SMILES: [N-]=[N+]=Nc1ccc(COC(=S)Nc2ccc(F)cc2)cc1] |

In some embodiments, the hydrogen sulfide-releasing compound is selected from the group consisting of morpholin-4-ium (4-methoxyphenyl)(morpholino)phosphinodithioate (GYY 4137, structure shown below), diallyl trisulfide (DATS, structure shown below), M₂S, MHS, and MS, wherein M is an alkali metal or an alkali earth metal.

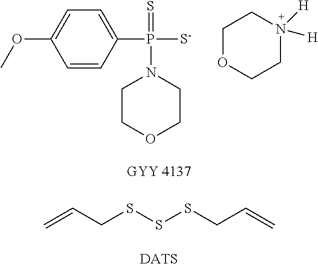

In some embodiments, the hydrogen sulfide-releasing compound is selected from the group consisting of morpholin-4-ium (4-methoxyphenyl)(morpholino)phosphinodithioate and diallyl trisulfide, or a pharmaceutically acceptable salt of diallyl trisulfide. In some embodiments, the hydrogen sulfide-releasing compound is morpholin-4-ium (4-methoxyphenyl)(morpholino)phosphinodithioate. In some embodiments, the hydrogen sulfide-releasing compound is diallyl trisulfide, or a pharmaceutically acceptable salt thereof. In some embodiments, the hydrogen sulfide-releasing compound is diallyl trisulfide.

In some embodiments, the hydrogen sulfide-releasing compound is selected from the group consisting of M₂S, MHS, and MS wherein M is an alkali metal or an alkali earth metal. In some embodiments, M is an alkali metal. In some embodiments, M is sodium or potassium. In some embodiments, M is sodium. In some embodiments, M is potassium. In some embodiments M is an alkali earth metal. In some embodiments, M is calcium. In some embodiments, the hydrogen sulfide-releasing compound is selected from the group consisting of Na₂S and K₂S. In some embodiments, the hydrogen sulfide-releasing compound is selected from the group consisting of NaHS and KHS. In some embodiments, the hydrogen sulfide-releasing compound is CaS.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the hydrogen sulfide-releasing compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

### Combination Therapies

One or more additional therapeutic agents such as, for example, anesthetics (e.g., for use in combination with a surgical procedure) or mydriatic agents may be administered in combination with the hydrogen sulfide-releasing compound, or pharmaceutically acceptable salt thereof, provided herein.

Exemplary anesthetics include, but are not limited, to local anesthetics (e.g., lidocaine, procain, ropivacaine) and general anesthetics (e.g., desflurane, enflurane, halothane, isoflurane, methoxyflurane, nitrous oxide, sevoflurane, mmobarbital, methohexital, thiamylal, thiopental, diazepam, lorazepam, midazolam, etomidate, ketamine, propofol, alfentanil, fentanyl, remifentanil, buprenorphine, butorphanol, hydromorphone levorphanol, meperidine, methadone, morphine, nalbuphine, oxymorphone, pentazocine).

Exemplary mydriatic agents include, but are not limited to, tropicamide, atropine, phenylephrine, and cyclopentolate.

In some embodiments, the additional therapeutic agent is administered simultaneously with a hydrogen sulfide-releasing compound, or pharmaceutically acceptable salt, provided herein. In some embodiments, the additional therapeutic agent is administered after administration of a hydrogen sulfide-releasing compound, or pharmaceutically acceptable salt, provided herein. In some embodiments, the additional therapeutic agent is administered prior to administration of a hydrogen sulfide-releasing compound, or pharmaceutically acceptable salt, provided herein.

In some embodiments, the hydrogen sulfide releasing-compound, or pharmaceutically acceptable salt thereof, is administered during a surgical procedure. In some embodiments, the hydrogen sulfide releasing-compound, or pharmaceutically acceptable salt thereof, is administered in combination with an additional therapeutic agent during a surgical procedure. In some embodiments, the surgical procedure is a surgical procedure for treating a cataract in the subject.

In some embodiments, the hydrogen sulfide releasing-compound, or pharmaceutically acceptable salt thereof, is administered before a surgical procedure. In some embodiments, the hydrogen sulfide releasing-compound, or pharmaceutically acceptable salt thereof, is administered in combination with an additional therapeutic agent before a surgical procedure.

For example, the hydrogen sulfide releasing-compound, or a pharmaceutically acceptable salt thereof, may be administered either alone or with an additional therapeutic agent about 30 seconds to about 7 days prior to the surgical procedure, e.g., about 30 seconds to about 5 days, about 30 seconds to about 3 days, about 30 seconds to about 24 hours, about 30 seconds to about 12 hours, about 30 seconds to about 6 hours, about 30 seconds to about 2 hours, about 30 seconds to about 1 hour, about 1 hour to about 7 days, about 1 hour to about 5 days, about 1 hour to about 5 days, about 1 hour to about 3 days, about 1 hour to about 24 hours, about 1 hour to about 12 hours, about 1 hour to about 6 hours, about 1 hour to about 2 hours, about 2 hours to about 7 days, about 2 hours to about 5 days, about 2 hours to about 5 days, about 2 hours to about 3 days, about 2 hours to about 24 hours, about 2 hours to about 12 hours, about 2 hours to about 6 hours, about 6 hours to about 7 days, about 6 hours to about 5 days, about 6 hours to about 5 days, about 6 hours to about 3 days, about 6 hours to about 24 hours, about 6 hours to about 12 hours, about 12 hours to about 7 days, about 12 hours to about 5 days, about 12 hours to about 5 days, about 12 hours to about 3 days, about 12 hours to about 24 hours, about 24 hours to about 7 days, about 24 hours to about 5 days, about 24 hours to about 5 days, about 24 hours to about 7 days, about 24 hours to about 5 days, about 24 hours to about 3 days, about 3 days to about 7 days, about 3 days to about 5 days, or about 5 days to about 7 days prior to the surgical procedure.

In some embodiments, the hydrogen sulfide releasing-compound, or pharmaceutically acceptable salt thereof, is administered after a surgical procedure. In some embodiments, the hydrogen sulfide releasing-compound, or pharmaceutically acceptable salt thereof, is administered in combination with an additional therapeutic agent after a surgical procedure. For example, the hydrogen sulfide releasing-compound, or a pharmaceutically acceptable salt thereof, may be administered either alone or with an additional therapeutic agent about 30 seconds to about 7 days after the surgical procedure, e.g., about 30 seconds to about 5 days, about 30 seconds to about 3 days, about 30 seconds to about 24 hours, about 30 seconds to about 12 hours, about 30 seconds to about 6 hours, about 30 seconds to about 2 hours, about 30 seconds to about 1 hour, about 1 hour to about 7 days, about 1 hour to about 5 days, about 1 hour to about 5 days, about 1 hour to about 3 days, about 1 hour to about 24 hours, about 1 hour to about 12 hours, about 1 hour to about 6 hours, about 1 hour to about 2 hours, about 2 hours to about 7 days, about 2 hours to about 5 days, about 2 hours to about 5 days, about 2 hours to about 3 days, about 2 hours to about 24 hours, about 2 hours to about 12 hours, about 2 hours to about 6 hours, about 6 hours to about 7 days, about 6 hours to about 5 days, about 6 hours to about 5 days, about 6 hours to about 3 days, about 6 hours to about 24 hours, about 6 hours to about 12 hours, about 12 hours to about 7 days, about 12 hours to about 5 days, about 12 hours to about 5 days, about 12 hours to about 3 days, about 12 hours to about 24 hours, about 24 hours to about 7 days, about 24 hours to about 5 days, about 24 hours to about 5 days, about 24 hours to about 7 days, about 24 hours to about 5 days, about 24 hours to about 3 days, about 3 days to about 7 days, about 3 days to about 5 days, or about 5 days to about 7 days after the surgical procedure.

### Pharmaceutical Formulations

When employed as pharmaceuticals, the compounds and salts provided herein can be administered in the form of pharmaceutical compositions. These compositions can be prepared as described herein or elsewhere, and can be administered by a variety of routes, depending upon whether local or systemic treatment is desired and upon the area to be treated. In some embodiments, the administration is topical administration (e.g. ophthalmic administration, administration to mucous membranes, and the like), oral, or parenteral administration (e.g., intravenous, intraarterial, subcutaneous, intraperitoneal, intramuscular or injection or infusion, intracranial, intrathecal, intraventricular administration, and the like). In some embodiments, administration is topical administration. In some embodiments, administration is ophthalmic administration. In some embodiments, administration is parenteral administration. In some embodiments, administration is oral administration.

Pharmaceutical compositions and formulations for topical administration may include ointments, creams, gels, drops, sprays, liquids, and powders. Conventional pharmaceutical carriers, aqueous, powder or oily bases, thickeners and the like may be necessary or desirable.

Also provided are pharmaceutical compositions which contain, as the active ingredient, a compound provided herein, or a pharmaceutically acceptable salt thereof, in combination with one or more pharmaceutically acceptable carriers (e.g., excipients). In making the compositions provided herein, the active ingredient is typically mixed with an excipient, diluted by an excipient. When the excipient serves as a diluent, it can be a solid, semi-solid, or liquid material, which acts as a vehicle, carrier or medium for the active ingredient. Thus, exemplary forms of the compositions include, but are not limited to, powders, elixirs, suspensions, emulsions, solutions, syrups, aerosols (as a solid or in a liquid medium), and ointments.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; or combinations thereof.

The active ingredient (e.g., the hydrogen sulfide-releasing compound provided herein) can be effective over a wide dosage range and is generally administered in a pharmaceutically effective amount. In some embodiments, the pharmaceutical composition comprises the active ingredient in an amount of from about 0.0001% to about 15%, for example, about 0.0001% to about 10%, about 0.0001% to about 5%, about 0.0001% to about 2%, about 0.0001% to about 1%, about 0.0001% to about 0.01%, about 0.01% to about 15%, about 0.01% to about 10%, about 0.01% to about 5%, about 0.01% to about 2%, about 0.01% to about 1%, about 1% to about 15%, about 1% to about 10%, about 1% to about 5%, about 1% to about 2%, about 2% to about 15%, about 2% to about 10%, about 2% to about 5%, about 5% to about 15%, about 5% to about 10%, or about 10% to about 15%. In some embodiments, the pharmaceutical composition comprises the active ingredient in an amount of from about 0.0001% to about 5%. In some embodiments, the pharmaceutical composition comprises the active ingredient in an amount of from about 1% to about 5%.

It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

### EXAMPLES

The slow H₂S releasing, water soluble prodrug, morpholin-4-ium-4-methoxyphenyl (morpholino) phosphinodithioate] (GYY 4137; see e.g., Li et al, Circ. 2008, 117; 2351-2360) and the polysulfides present in garlic, diallyl trisulfide (DATS; see e.g., Proceedings of the Nat. Acad. Sci. USA, 2007, 104(46):17977-17982) were purchased from Cayman Chemical Co., Ann Arbor, Mich.; Stock solutions of GYY 4137 and DATS were prepared in dimethyl sulfoxide (DMSO) according to the product specifications.

### Example 1. Effects of H₂S-Releasing Compounds on Cataract Formation in Cultured Bovine Lenses

GYY 4137 has been established as a slow-releasing H₂S donor, in vitro and in vivo (see e.g., Li et al, Circ. 2008, 117:2351-2360; and Whiteman et al, Antioxid. Redox Signal, 2010, 12:1147-1154) and was used to measure the effects of an H₂S-releasing compound on cataract formation in cultured bovine lenses. Briefly, freshly isolated bovine eyes were transported from a local slaughterhouse in an ice bucket. Lenses were carefully removed and cultured in 12-well plates in DMEM buffer containing antibiotics in the absence (control) or presence of either H₂O₂ (50 mM), GYY 4137 (10⁻⁴ M) only or H₂O₂ plus GYY 4137. Lenses were then incubated in a carbon dioxide chamber at 37° C., photographed, and assessed for light transmittance (wavelength 230-710 nm) at times 0, 3, 6, 12 hours and every 12 hours up to 120 hours using a plate reader (Synergy H1 hybrid reader, Bio Tek Instruments, Inc., Winooski, Vt.). All measurements were expressed as percent transmittance of the zero-hour, medium-treated lenses.

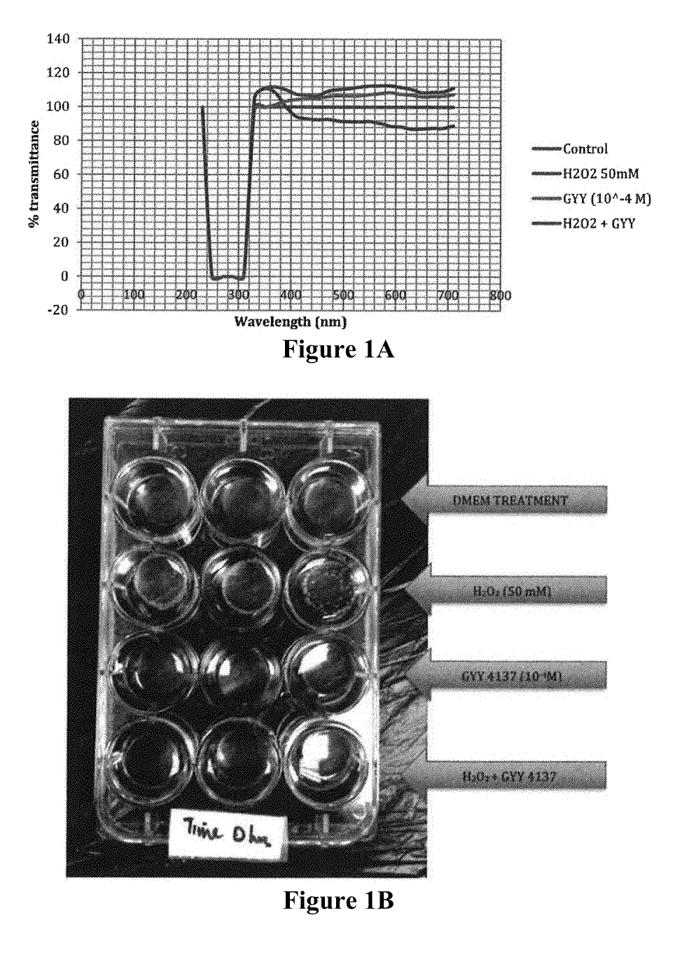

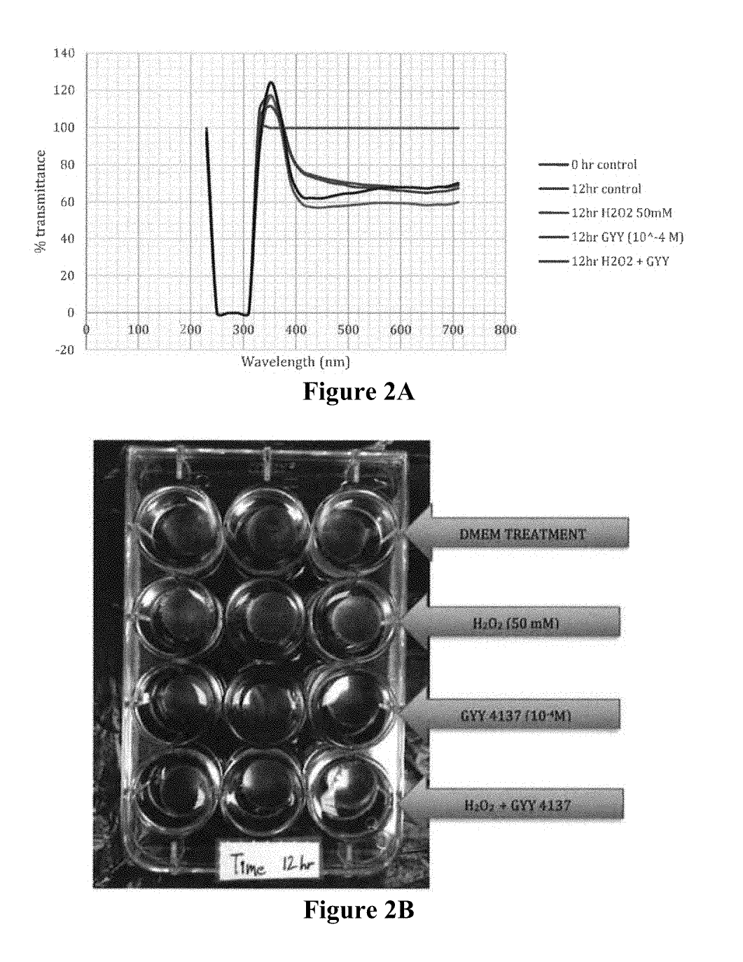

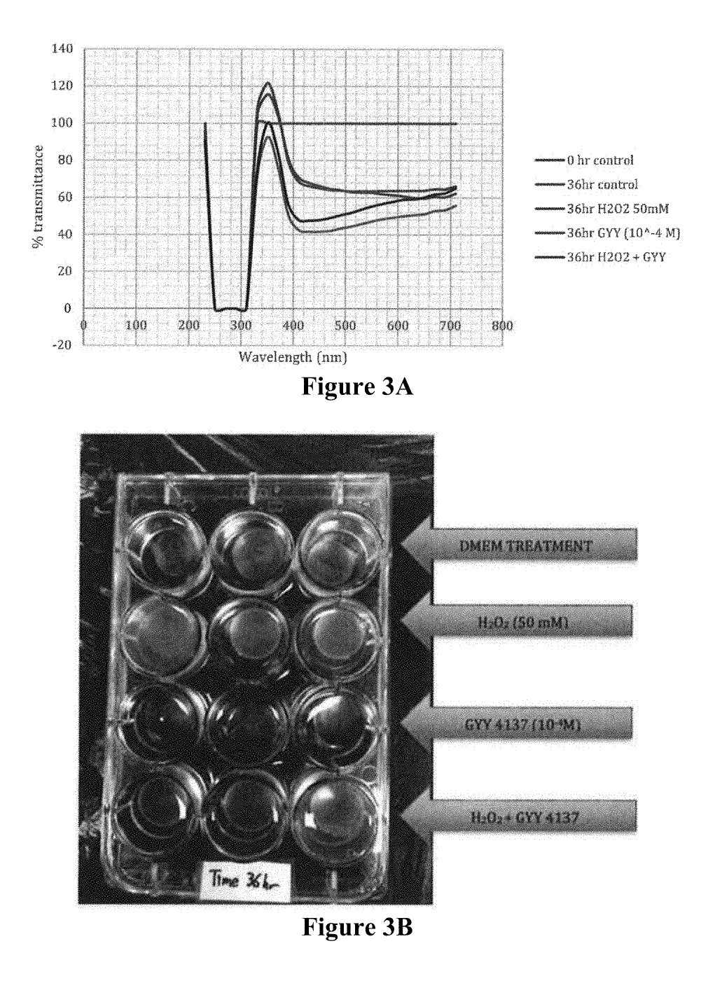

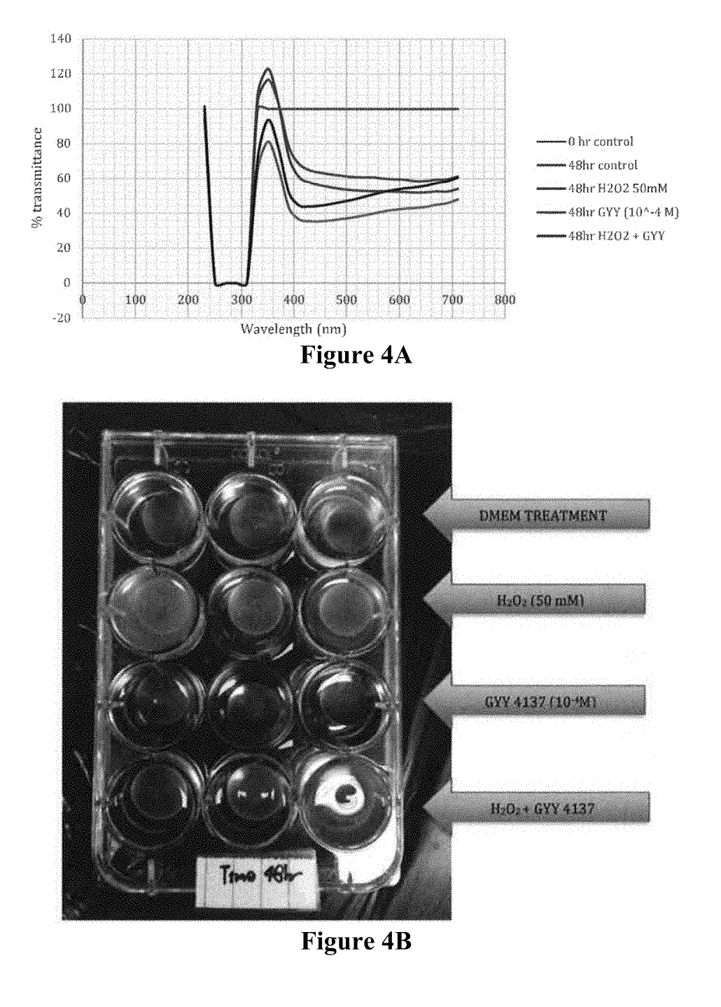

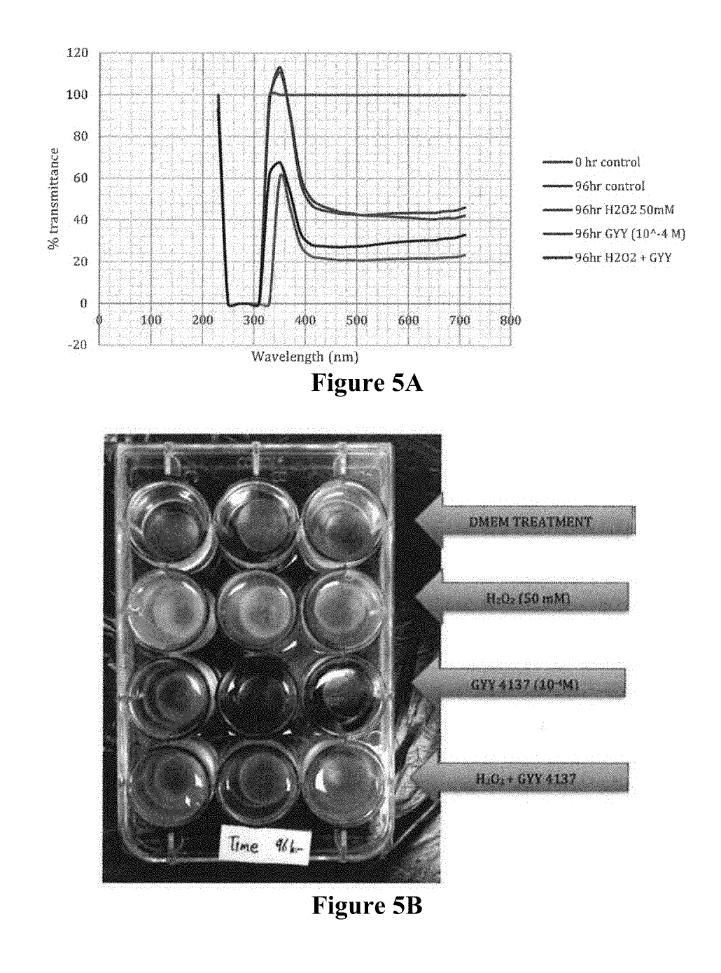

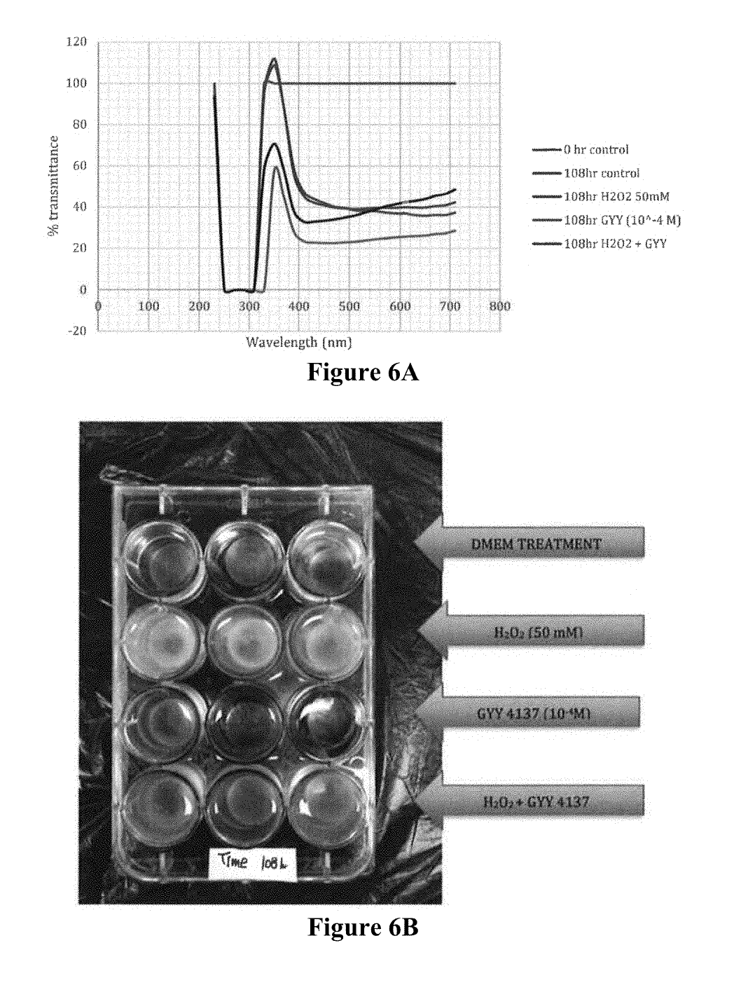

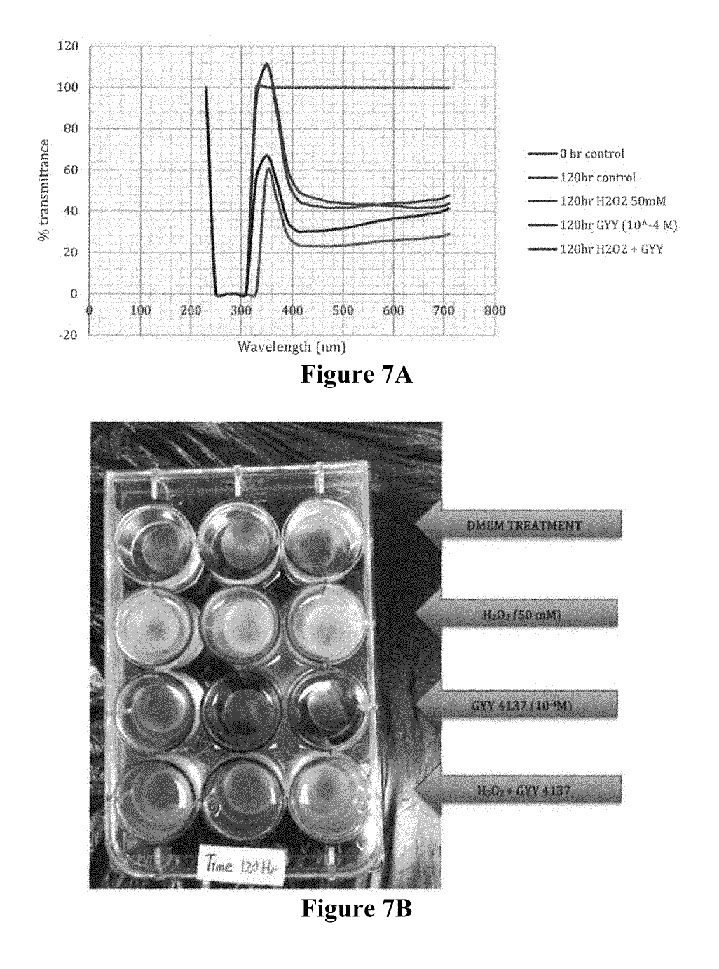

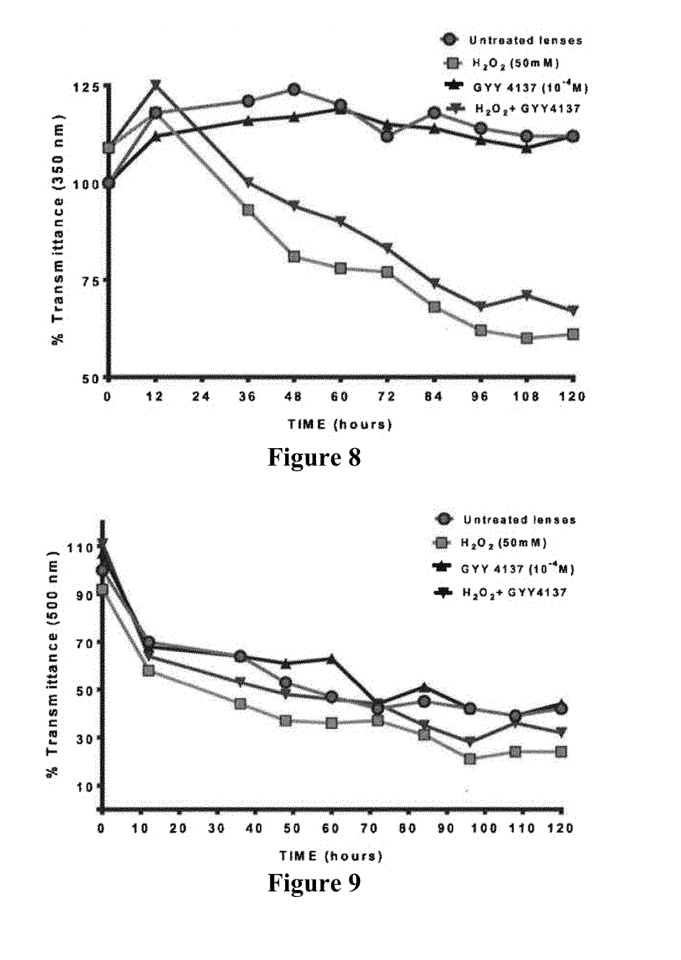

As shown in FIGS. 1-9, GYY 4137 (10⁻⁴ M) delayed the progression of cataract formation in medium-treated lenses for up to 60 hours. For example, at the 60-h time point, the H₂S donor attenuated transmittance by 22% at the 500 nm wavelength. Interestingly, GYY 4137 (10⁻⁴ M) attenuated the progression of H₂O₂-induced opacification for the entire period (120 hours) of study, as shown in FIGS. 1A-9. FIGS. 8 and 9 depict the % transmittance at 350 and 500 nm, respectively. Without being bound by theory, these data suggest that GYY 4137, and other H₂S donors, have a potential therapeutic application in management of cataracts.

### Example 2. Experimental Model of Lens Degradation of Untreated Lenses

### Cultured Bovine Lenses

Freshly isolated bovine eyes were obtained and transported into the laboratory in an ice bucket within three hours of enucleation. Eyeballs were dissected in an ice bath and lenses were extracted and kept in refrigerated, ice cold Krebs buffer solution for further studies. Lenses were placed in each well (12-well plate) containing Dulbecco's Modified Eagle's medium (DMEM) in the absence (control) or presence of either H₂O₂; Test compound (GYY 4137; DATS) only or hydrogen peroxide (H₂O₂) plus test compound. To prevent microbial proliferation, a combination of penicillin (10,000 IU/mL) and streptomycin (10,000 μg/mL; 5 mL per 500 mL DMEM) was added to culture medium. Lenses were then incubated in a carbon dioxide (CO₂) chamber (Napco e series CO₂ incubator model 5100) at 37° C. (5% CO₂). Each well was replenished with respective treatment medium every 24 hours.

To determine the optimal concentration of H₂O₂ required for induction of cataract, lenses were treated with 5 mM, 10 mM, 50 mM and 100 mM of the peroxide. Based upon quantitative and qualitative evaluation, H₂O₂ (50 mM) was chosen for further studies. The endogenous antioxidant, ascorbic acid (3 mM; 10 mM) was used as a positive control. The preventive effect of H₂S releasing compounds on cataract formation was determined by culturing bovine lenses in DMEM containing either of H₂S donors, GYY4137 (10⁻⁷M to 10⁻³M) or DATS (10⁻⁷ to 10⁻⁴M). The ability of H₂S releasing compounds to protect bovine lenses from H₂O₂-induced cataract formation was determined by exposing lenses to H₂S donors, GYY 4137 (10⁻⁷M to 10⁻³M) and DATS (10⁻⁷ to 10⁻⁴M) in the presence of H₂O₂ (50 mM). Lens opacity was evaluated quantitatively and qualitatively at specific time intervals up to 120 hours.

### Assessment of Opacity

Quantitative evaluation of cataract formation was achieved using a plate reader (Synergy H1 hybrid reader; Bio Tek Instruments, Inc). Light transmittance was assessed between 230 nm and 710 nm at 40 nm intervals. In initial experiments, light transmittance of untreated lenses was compared at 0-hour and 120 hours at different wavelengths. It was observed that light transmittance at 420 nm exhibited the greatest change in magnitude (38%), compared to 460 nm (33.9%), 500 (30.97%), 540 (28.8%) and 580 nm (27.21%) following 120 hours incubation. This pattern of response was consistent throughout the experiments. Therefore, subsequent data was generated as transmittance at the 420 nm.

A decrease in transmittance indicated opacity and formation of cataract while an increase in transmittance or no change from initial measurement (time=0 hours) was considered as evidence of protection against cataract formation. Light transmittance was measured at times 0, 3, 6 hours on the first day after which measurements were conducted every 24 hours up to 120 hours. All measurements were expressed as either absolute transmittance or % transmittance of either the zero-hour or 120 hours, medium-treated lenses (control).

Qualitative evaluation of cataract formation was achieved by visual inspection and photographical images captured against a black grid, a standard method of assessment for cataract formation (see e.g., Ruiz-Ederra and Verkman, Invest. Ophthalmol. Vis. Sci. 2006, 47(9):3960-3967). Picture captures were conducted at the same time intervals as that of the quantitative assessment.

### Example 3. Development of Experimental Model of Lens Degradation of Untreated Lenses

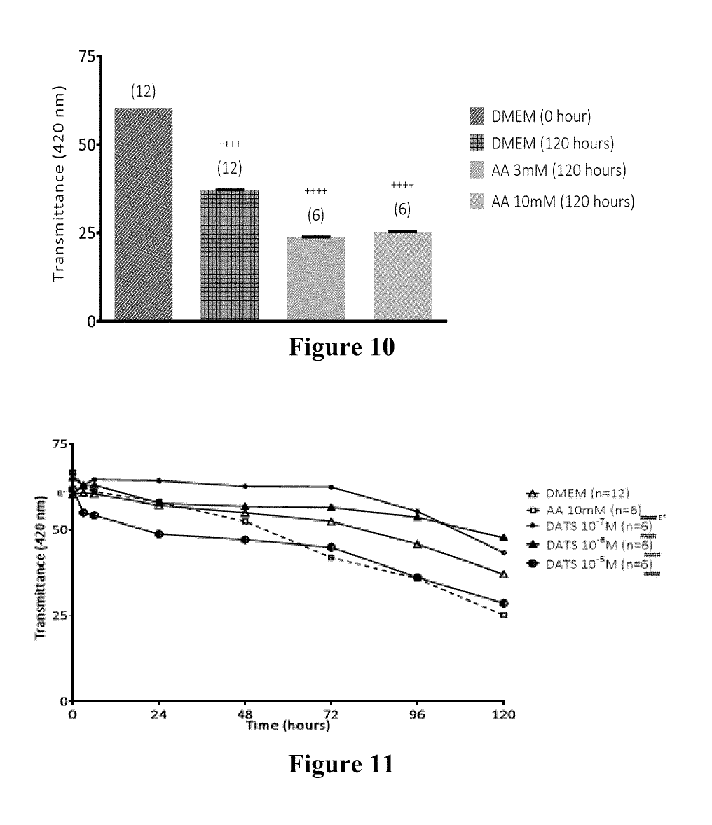

As illustrated in FIG. 10, untreated, DMEM cultured bovine lenses (time=0 hours) elicited light transmittance of 60.36±0.033 at 420 nm wavelength. After 120 hours, the same lenses exhibited significant reduction of light transmittance of 38.57% (p<0.0001; n=12). Similarly, the presence of the endogenous antioxidant (3 mM and 10 mM) significantly (p<0.0001; n=6) attenuated transmittance by 60.57% and 57.42%, respectively compared to the untreated, DMEM lenses (time=0 hours). These observations were corroborated by the qualitative findings, which showed that after 120 hours DMEM cultured bovine lenses exhibited less clarity, with the dark gridlines being less visible (FIG. 13, Top Panel). Moreover, the presence of ascorbic acid accelerated the degradation process, with the lenses becoming almost completely opaque after 120 hours (FIG. 13, Top Panel; ascorbic acid (10 mM) is shown in the picture). These observations suggest a time-dependent degradation of DMEM-cultured bovine lenses that is unresponsive to ascorbic acid (3 mM and 10 mM), in vitro. This approach was used as the experimental model for assessment of cataract formation in cultured bovine lenses, in vitro.

### Example 4. Protective Effect of H₂S Releasing Compounds on Lens Degradation

### Effect of DATS on Lens Degradation

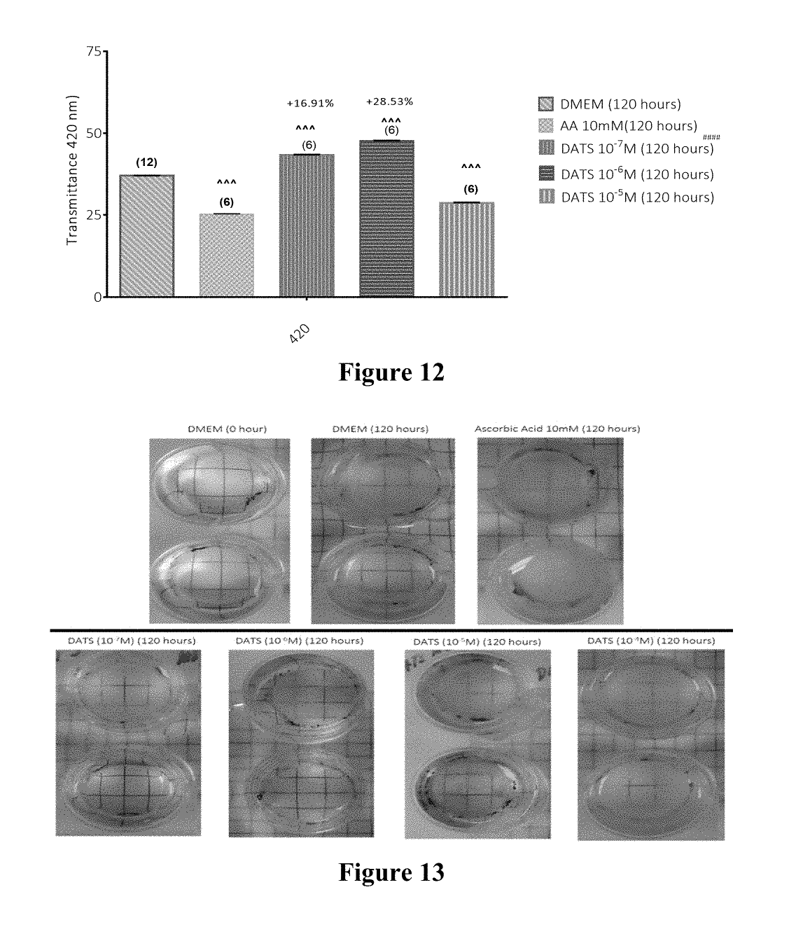

The effect of DATS (10⁻⁷M to 10⁻⁴M) on bovine lens degradation over a period of 120 hours was then measured. DATS is a polysulfide found in garlic that has been shown to release H₂S (see e.g., Benavides et al, Proceedings of the Nat. Acad. Sci. USA, 2007, 104(46):17977-17982). This polysulfide (10⁻⁷ and 10⁻⁶M) increased light transmittance up to 120 hours (FIG. 11). Compared to untreated DMEM cultured lenses (120 hours) the lower concentrations of DATS (10⁻⁷M and 10⁻⁶M) significantly (p<0.0001; n=6) improved light transmittance by 16.91% and 28.53%, respectively (FIG. 12). Moreover, this effect was superior to that of the endogenous antioxidant, ascorbic acid (10 mM) and that of the higher concentrations of DATS (10⁻⁵M) at the 120 hours-time point.

In accordance with the quantitative observations, DATS (10⁻⁷M and 10⁻⁶M) decelerated lens degradation as evidenced by the visibility of the dark gridlines (FIG. 13, Bottom Panel) after 120 hours. On the contrary, ascorbic acid (10 mM) and DATS (10⁻⁵M) accelerated lens degradation within the same time range (FIG. 13, Top and Bottom Panels). Taken together, this data suggests that lower concentrations of this H₂S donating polysulfide could play a preventive role in cataract formation.

### Effect of GYY 4137 on Lens Degradation

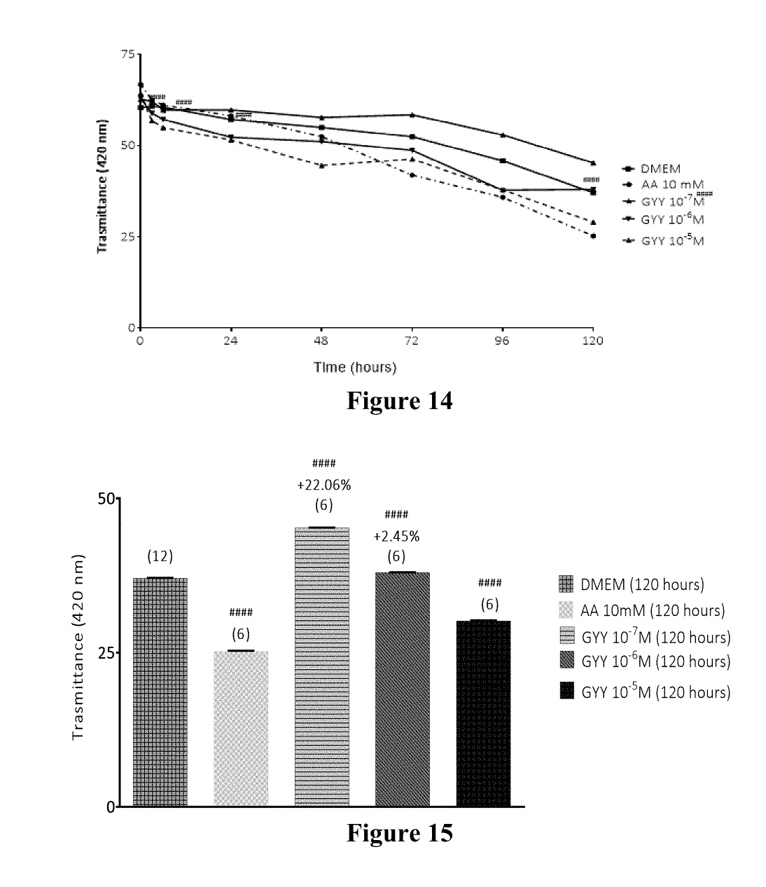

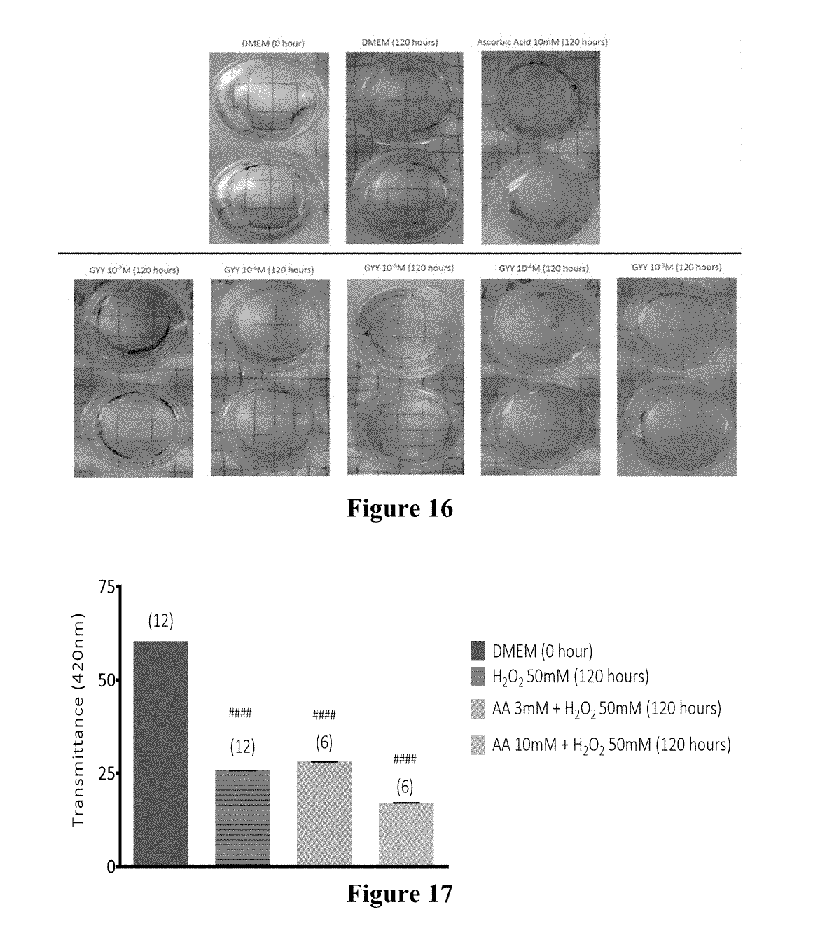

The effect of the slow releasing H₂S donor, GYY 4137 (see e.g., Li et al, Circ. 2008, 117:2351-2360) (10⁻⁷M to 10⁻⁵M) on the degradation of cultured bovine lenses was then measured. The lower concentration of GYY 4137 (10⁻⁷M) increased light transmittance at 420 nm up to 120 hours (FIG. 14). Compared to untreated lenses, GYY 4137 (10⁻⁷M & 10⁻⁶M) significantly (p<0.0001) increased transmittance by 22.06% and 2.45%, respectively at the 120-hour time point (FIG. 15). This effect was superior to that elicited by ascorbic acid (10 mM) and GYY 4137 (10⁻⁵M) at the same time point. Congruent to the quantitative data, GYY 4137 (10⁻⁷M) was most effective at maintaining clarity of the lenses after 120 hours (FIG. 16, Bottom Panel). Moreover, ascorbic acid (10 mM) and higher concentrations of GYY 4137 accelerated clouding of the lens within the same time range (FIG. 16, Top and Bottom Panels).

In summary, the lower concentrations of H₂S donors, DATS, and GYY 4137 maintained lens clarity up to 120 hours and were more potent than the endogenous antioxidant, ascorbic acid. The rank order of activity was as follows: DATS 10⁻⁶M>GYY 10⁻⁷M>DATS 10⁻⁷M>GYY 10⁻⁶M>AA 10 mM. Taken together, these results suggest a preventive effect of H₂S donors on lens opacification/degradation.

### Example 5. Effect of H₂S Releasing Compounds on H₂O₂-Induced Lens Degradation

The effect of H₂S releasing compounds on H₂O₂-induced cataract formation was then measured. Oxidative stress has been implicated in the pathogenesis of cataract development in the mammalian eye (see e.g., Sakamoto et al, Exp. Eye Res. 2014, 120:90-96; Osborne et al, Invest. Ophthalmol. & Vis. Sci. 2012, 51:284-294; and Spector et al, FASEB J. 1995, 9:1173-1182). Moreover, H₂O₂ content was found to be significantly elevated in aqueous humor and lenses of cataract patients (see e.g., Sakamoto et al, Exp. Eye Res. 2014, 120:90-96). Thus, H₂O₂ insult was used to induce cataract formation in cultured bovine lenses.

Compared to DMEM-cultured bovine lenses (time=0 hour), H₂O₂ (50 mM) decreased light transmittance by 36.76% after 120 hours. Whereas ascorbic acid (3 mM) attenuated H₂O₂-induced attenuated this inhibition by 9.22%, ascorbic acid (10 mM) failed to protect from peroxide-induced lens degradation (FIG. 17). This experimental approach was used as the model for peroxide-induced cataract formation, with ascorbic acid (3 mM) serving as a positive control.

DATS Protection from H₂O₂-Induced Lens Degradation

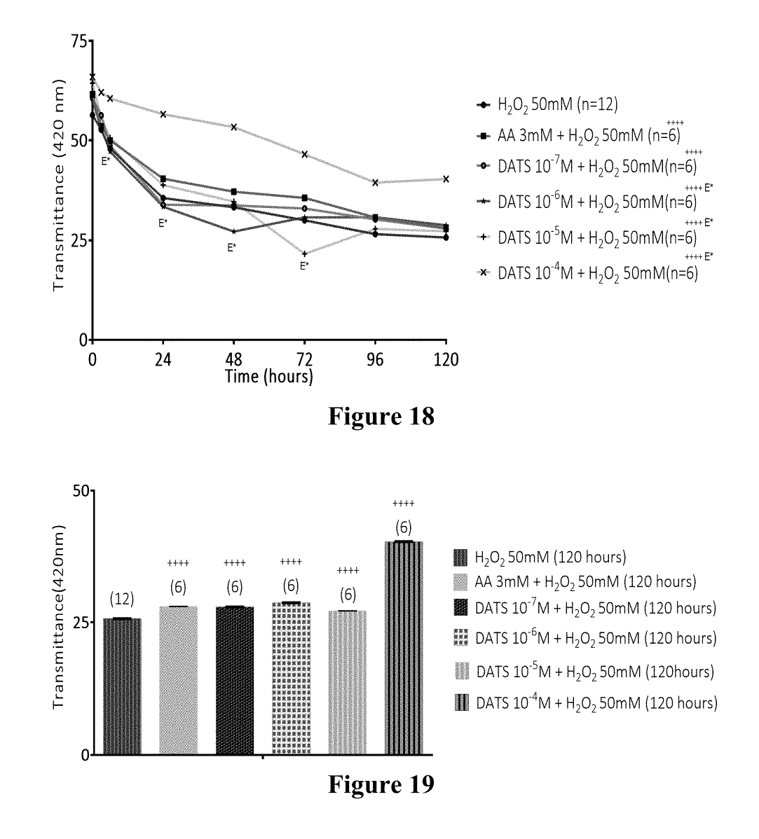

As demonstrated in FIGS. 18-19, DATS (10⁻⁷M to 10⁻⁴M) increased light transmittance of cultured bovine lenses in presence of H₂O₂, compared to H₂O₂ (50 mM) only. After 120 hours, DATS conferred significant (p<0.0001) protection as follows: 10⁻⁷M (8.83%) 10⁻⁶M, (11.95%), 10⁻⁵M (5.99%) and 10⁻⁴M (56.89%) (FIG. 19). The protective effect of ascorbic acid (3 mM) was comparable to that of the lower concentrations of DATS. However, the highest concentration of DATS (10⁻⁴M) elicited the highest (56.89%) protective potency from peroxide-induced degradation.

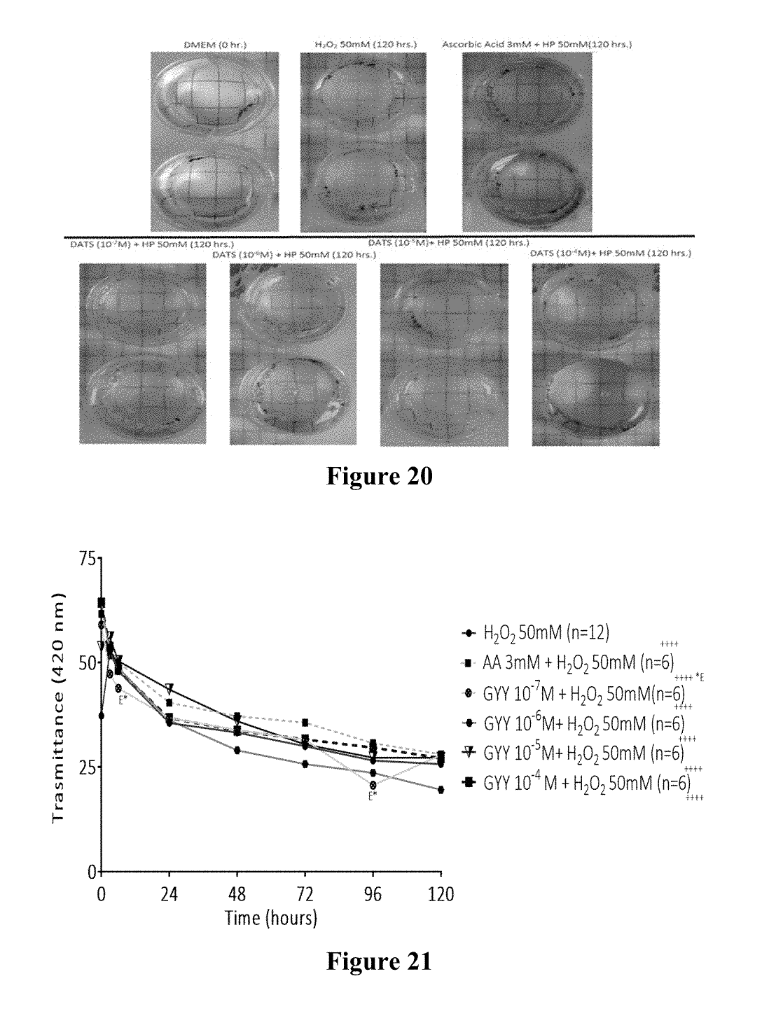

Similar to the quantitative data, DATS and ascorbic acid (3 mM) protected lenses from peroxide-induced degradation, with the DATS (10⁻⁴M) exhibiting the highest level of protection (FIG. 20). Taken together, these data suggesting a protective role for this H₂S donor on peroxide-induced lens degradation and it is believed that DATS could play a protective role in oxidant stress-induced cataract formation.

GYY 4137 Protection Against H₂O₂-Induced Lens Degradation

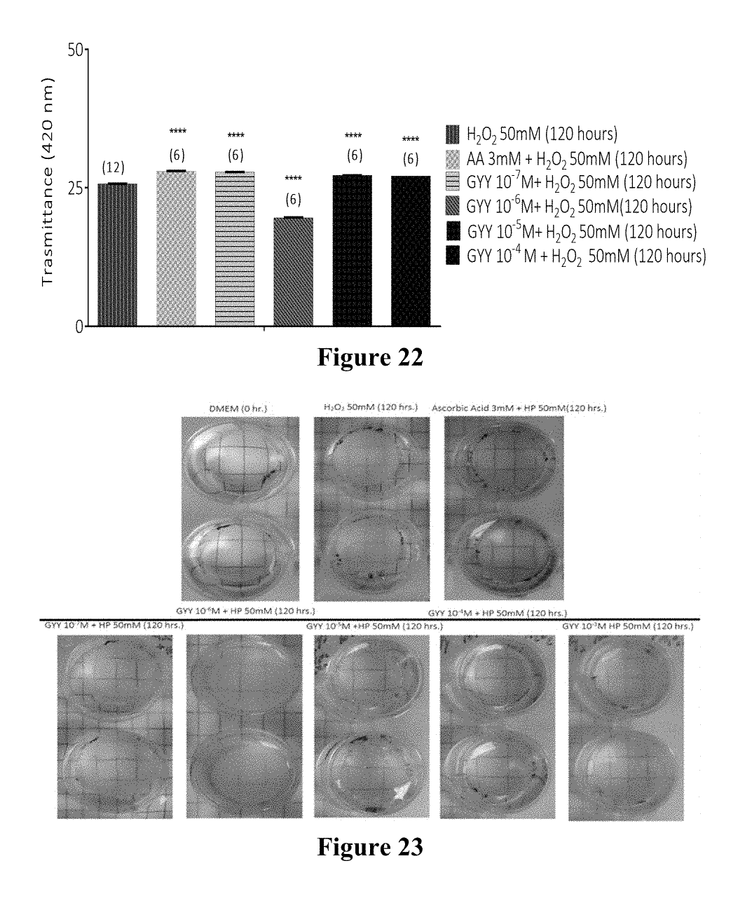

The slow H₂S releasing compound, GYY 4137 (10⁻⁷M, 10⁻⁵M & 10⁻⁴M) protected cultured bovine lenses from H₂O₂-induced degradation for up to 120 hours, by significantly (p<0.0001) increasing transmittance (420 nm) by 8.4% (10⁻⁷M), 6.03% (10⁻⁵M), and 5.56% (10⁻⁴M) (FIGS. 21-22). The protective action of GYY 4137 was comparable to that of ascorbic acid (3 mM). Interestingly, GYY 4137 (10⁻⁶M & 10⁻³M) concentrations did not provide any protection against H₂O₂-induced degradation of the lenses. Whereas GYY 4137 (10⁻⁷M) exerted a superior protective action against lens degradation (FIG. 14-15), it was not equally potent against H₂O₂-induced lens degradation. As illustrated by FIG. 23, all lenses treated with GYY 4137 (10⁻⁷M, 10⁻⁵M & 10⁻⁴M) displayed relatively clear grids after 120 hours, compared to the untreated lenses. Similar to DATS, these data suggest a protective role for GYY 4137 from oxidant-induced cataract formation.

In summary, DATS and GYY 4137 protected lenses against H₂O₂ (50 mM)-induced degradation for up to 120 hours. With the exception of DATS (10⁻⁴M), these affects were comparable to that of the endogenous antioxidant, ascorbic acid (3 mM). Since oxidative stress is an underlying pathology in the pathogenesis of cataracts, these data suggest that H₂S releasing compounds may have a role in slowing down the progression of cataract formation.

### Example 6. Biochemical Change Due to Treatment with H₂S Producing Compounds

The following biochemical assays were conducted to measure changes in glutathione (GSH) and superoxide dismutase (SOD) enzyme content following lens opacification/degradation after 120 hours.

### Glutathione Activity

Lenses were rinsed with phosphate buffered saline solution (pH 7.4) and 1 g weight of lens was homogenized in 10 mL of cold phosphate buffer (pH 6.7) containing EDTA (1 mM). Tissue homogenate were centrifuged (Sorvall Legend RT) at 4000×g for 5 mins at 4° C. and the supernatant was collected and stored at ice-cold temperature for deprotonation. Deprotonation was achieved by dissolving metaphosphoric acid (5 g; Sigma-Aldrich) in 50 mL of water. The metaphosphoric acid solution was then added to the sample in a 1:1 ratio and vortexed. The mixture was kept at room temperature for 5 minutes, then centrifuged at 4000 g for at least two minutes. The supernatant, which contained glutathione (GSH) and glutathione disulfide dimer (GSSG) was collected without disturbing the precipitate and stored at −20° C. for GSH assay. 50 μL of triethanolamine (TEAM reagent; 4M) per 1 mL of the supernatant were mixed and vortexed immediately. Further dilutions were accomplished using GSH mycothiol (MES) buffer solution and total GSH (both reduced and oxidized) was then measured following the GSH assay kit (Cayman Chemical, Item no. 703002) protocol. The absorbance was measured at 405 nm using a plate reader (Synergy H1 hybrid reader; Bio Tek Instruments, Inc).

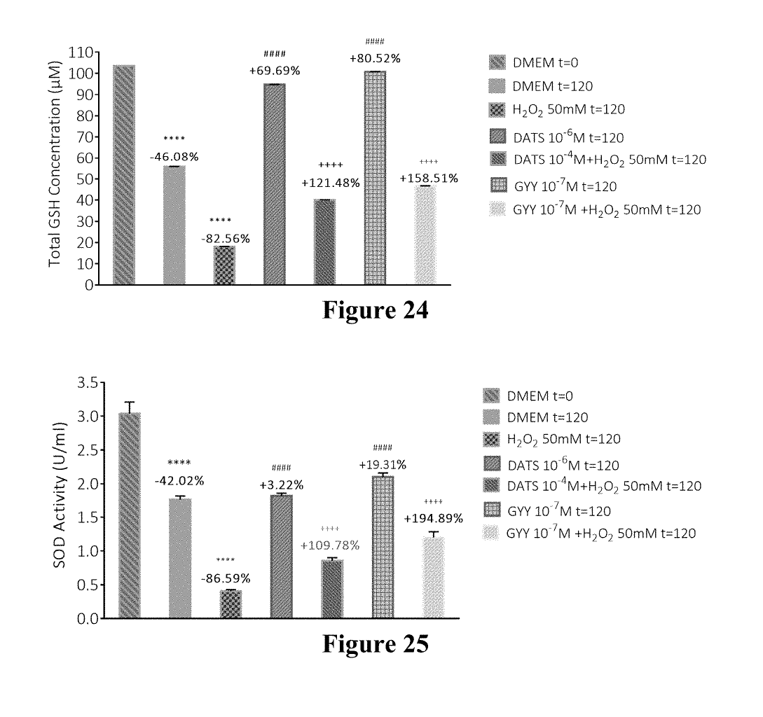

There is evidence that H₂S exerts a protective action on neurons by replenishing intracellular glutathione stores (see e.g., Kimura and Kimura, FASEB J. 2004, 18:1165-1167; and Kimura et al, Antioxid. Redox. Signal 2010, 12:1-13). Therefore, in the next series of experiments, the effect of H₂S releasing compounds on the GSH activity in cultured bovine lenses was examined. It was observed that the total lenticular GSH content in DMEM-cultured bovine lenses decreased significantly (p<0.0001) by 46.08% over the duration of 120 hours, compared to untreated lens (time=0 hour). Moreover, the presence of H₂S releasing compounds, DATS (10⁻⁶M) and GYY 4137 (10⁻⁷M), significantly (p<0.0001) reversed this decline in GSH content by of 69.69% and 80.52%, respectively (FIG. 24), supporting the preventive function for these H₂S releasing compounds from lens degradation.

The presence of H₂O₂ (50 mM) attenuated GSH content by 82.56%, compared to untreated lenses (time=0 hours), confirming that oxidant stress accelerates lens degradation. The presence of DATS (10⁻⁶M) and GYY 4137 (10⁻⁷M) in H₂O₂-treated lenses significantly (p<0.0001) increased GSH content by 121.48% and 158.51%, respectively, after 120 hours, affirming the protective function of these H₂S donors in peroxide-induced lens degradation (FIG. 24).

### Superoxide Dismutase Activity

Lenses were rinsed with phosphate buffered saline solution (pH 7.4) and 1 g weight of lens homogenized in cold HEPES buffer (20 mM; pH 7.2) containing EGTA (1 mM), mannitol (210 mM) and sucrose (70 mM). The tissue homogenate was then centrifuged at 1500×g for 5 minutes at 4° C. The supernatant was collected over ice and stored at −80° C. This supernatant, which contained both cytosolic and mitochondrial superoxide dismutase (SOD) was assayed using the SOD assay kit (Cayman Chemical, Item no. 706002) protocol. SOD activity was determined by measuring absorbance at 440 nm using a plate reader (Synergy H1 hybrid reader; Bio Tek Instruments, Inc).

For both GSH and SOD assay, each treatment had a sample size of five. Lens homogenates were treated as follows:

Untreated lens t=0 hour

Untreated lens t=120 hours

H₂O₂ (50 mM) treated lens t=120 hours

DATS (10⁻⁶M) treated lens t=120 hours

GYY 4137 (10⁻⁷M) treated lens t=120 hour

DATS (10⁻⁴M)+H₂O₂ (50 mM) lens t=120 hours

GYY 4137 (10⁻⁷M)+H₂O₂ (50 mM) lens t=120 hours

Results are presented as arithmetic means±S.E.M. Significance of differences between control and treated groups was evaluated using one-way analysis of variance (ANOVA) followed by dunnett's multiple comparisons test (Graph Pad Prism 6). Differences with at least P<0.05 were considered statistically significant.

Superoxide dismutase (SOD) is an antioxidant enzyme that serves a protective function in tissues. To assess the antioxidant status of lenses, SOD content was measured following treatment with either H₂O₂, H₂S releasing compounds, or a combination of both. As demonstrated in FIG. 25, SOD content in DMEM-cultured bovine lenses decreased significantly (p<0.0001) by 42.06% over the duration of 120 hours, compared to untreated lens (0 hour). The presence of H₂S releasing compounds, DATS (10⁻⁶M) and GYY 4137 (10⁻⁷M) significantly (p<0.0001) reversed this decline in SOD content by 3.22% and 19.3%, respectively (FIG. 25), supporting the preventive function for H₂S releasing compounds on cataract formation.

The presence of H₂O₂ (50 mM) attenuated SOD content by 86.59%, compared to untreated lenses (time=0 hours) (FIG. 25), affirming the role of oxidant stress in the acceleration of cataract formation. DATS (10⁻⁶M) and GYY 4137 (10⁻⁷M) significantly (p<0.0001) reversed H₂O₂-mediated loss of SOD content by 109.78% and 194.89%, respectively, after 120 hours.

Both GSH and SOD content declined over the duration of 120 hours, corroborating the lens degradation observed in FIG. 10 and suggests that lens degradation is associated with loss of endogenous antioxidant capacity. The H₂S releasing compounds DATS and GYY 4137 significantly reversed the decline in GSH and SOD, suggesting that the loss of antioxidant integrity contributes to the degradation/opacification of the lens. Without being bound by theory, it is also theorized that H₂S donors confer prevention of lens degradation by replenishing the antioxidant integrity of the lens. H₂O₂ accelerated lens degradation, as evidenced by the magnitude in reduction of both GSH and SOD content, implying that this peroxide degrades the lens by depleting the endogenous antioxidant stores. Furthermore, this decline was significantly reversed by both DATS and GYY 4137.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of preventing a cataract in a subject in need thereof, comprising administering to the subject a therapeutically effective amount of a hydrogen sulfide-releasing compound, or a pharmaceutically acceptable salt thereof.

2. A method of delaying the formation of a cataract in a subject in need thereof, comprising administering to the subject a therapeutically effective amount of a hydrogen sulfide-releasing compound, or a pharmaceutically acceptable salt thereof.

3. The method of claim 2, wherein the method comprises delaying opacification of the eye or reducing the rate of opacification of the eyes.

4. The method of claim 3, wherein the opacification comprises H₂O₂-induced opacification.

5. A method of reducing oxidative stress in the eye of a subject in need thereof, comprising administering to the subject a therapeutically effective amount of a hydrogen sulfide-releasing compound, or a pharmaceutically acceptable salt thereof.

6. A method of monitoring the formation of a cataract in a subject in need thereof, comprising:
i) administering to the subject a therapeutically effective amount of a hydrogen sulfide-releasing compound, or a pharmaceutically acceptable salt thereof; and
ii) imaging an eye of the subject with a suitable imaging technique.

7. The method of claim 6, wherein the method further comprises imaging an eye of the subject with a suitable imaging technique prior to step i).

8. The method of claim 6, wherein the imaging is performed using a photographic imaging technique, a digital imaging technique, or any combination thereof.

9. The method of claim 1, wherein the method comprises locally administering the compound, or pharmaceutically acceptable salt thereof, to an eye of the subject.

10. The method of claim 1, wherein the compound, or pharmaceutically acceptable salt thereof, is administered via topical administration.

11. The method of claim 1, wherein the compound, or pharmaceutically acceptable salt, is administered in the form of a pharmaceutical composition comprising the compound, or pharmaceutically acceptable salt thereof, and at least one additional pharmaceutically acceptable carrier.

12. The method of claim 1, wherein the subject has been identified as exhibiting one or more symptoms associated with a cataract.

13. The method of claim 1, wherein the subject has been identified as exhibiting one or more risk factors associated with a cataract selected from the group consisting of diabetes, obesity, hypertension, ocular trauma, myopia, ocular inflammatory disease, and chronic use of corticosteroids.

14. The method of claim 1, wherein the hydrogen sulfide-releasing compound is selected from the group consisting of morpholin-4-ium (4-methoxyphenyl)(morpholino)phosphinodithioate, diallyl trisulfide, M₂S, and MHS, wherein M is an alkali metal.

15. The method of claim 14, wherein the hydrogen sulfide-releasing compound is selected from the group consisting of morpholin-4-ium (4-methoxyphenyl)(morpholino)phosphinodithioate and diallyl trisulfide.

16. The method of claim 14, wherein M is sodium or potassium.

17. The method of claim 1, wherein the method in performed in combination with a surgical procedure for treating a cataract.

18. The method of claim 1, wherein the method further comprises administering one or more additional therapeutic agents useful for the treatment of a cataract to the subject.

19. The method of claim 1, wherein the method comprises reducing and/or reversing the degradation of glutathione (GSH)

20. The method of claim 1, wherein the method comprises reducing and/or reversing the degradation of superoxide dismutase (SOD) enzyme in the subject.
